(19) United States
(12) Patent Application Publication
(10) Pub. No.: US 2022/0354779 A1
(43) Pub. Date: Nov. 10, 2022

(54) NANOFIBER- AND NANOWHISKER-BASED TRANSFECTION PLATFORMS

(71) Applicants: OHIO STATE INNOVATION FOUNDATION, Columbus, OH (US); NANOFIBER SOLUTIONS, LLC, HILLIARD, OH (US)

(72) Inventors: Daniel Gallego-Perez, Columbus, OH (US); Natalia Higuita-Castro, Columbus, OH (US); Jed Johnson, Hilliard, OH (US); Devan Ohst, Hilliard, OH (US)

(21) Appl. No.: 17/753,890

(22) PCT Filed: Sep. 21, 2020
(86) PCT No.: PCT/US2020/051785

§ 371 Date: Mar. 17, 2022

Related U.S. Application Data
(60) Provisional application No. 62/902,723, filed on Sep. 19, 2019.

(51) Int. Cl. A61K 9/00; A61K 9/48; A61K 38/17; A61K 38/39; A61K 38/18; A61K 9/70

(52) U.S. Cl. CPC A61K 9/0009; A61K 9/0014; A61K 9/0019; A61K 9/4816; A61K 9/485; A61K 38/1729; A61K 38/39; A61K 38/1808; A61K 9/4866; A61K 38/1866; A61K 9/7007

(57) ABSTRACT

Described herein are electrospun core-shell fibers that include (i) a central core that is electrically conductive having an exterior surface, wherein the core comprises a first polymer and an electroconductive material; (ii) a shell adjacent to the exterior surface of the core, the shell comprising a second polymer; and (iii) one or more bioactive agents in the shell. In one aspect, the fibers are electrospun fibers. Additionally, described herein are methods for making and using the core-shell fibers.

### CROSS REFERENCE TO RELATED APPLICATIONS

This application claims the benefit of U.S. Provisional Application No. 62/902,723, filed on Sep. 19, 2019, which is incorporated herein by reference in its entirety.

### TECHNICAL FIELD

The present disclosure generally relates to materials and compositions for therapeutic, prophylactic, or diagnostic applications such as wound healing and drug delivery.

### BACKGROUND

Wound healing refers to at least four highly integrated and overlapping phases: hemostasis, inflammation, proliferation, and tissue remodeling or resolution which must occur in the proper sequence, at a specific time, and continue for a specific duration at an optimal intensity. There are many factors that can affect wound healing which interfere with one or more phases in this process, thus causing improper or impaired tissue repair.

Promotion of wound healing remains the focus of intensive research and study and there are currently numerous methods and compositions available to treat wounds and promote wound healing, including a myriad of passive and active dressings and bandages, and topical medicaments, as well as physical and/or chemical debridement of necrotic tissue. Wound healing might also involve necrosis, apoptosis and alteration of the cell growth of non-transformed tissue. Despite this, results have been somewhat inconsistent and the treatment of chronic or slow healing wounds continues to pose a serious challenge for the medical fraternity.

Of particular concern, military wounds pose many inherent challenges, including high severity, multiplicity, and high rates of contamination with microorganisms and/or other elements from the environment (e.g., from improvised explosive devices). As such, in many cases, wound management has to go through all five echelons of medical care until successful evacuation to a major military center in the US2, 3. Given the complexity of military wounds, there is a growing push for forward implementation of more advanced wound care strategies at lower levels of care (I-II). Early measures to prevent severe contamination and/or other complications (e.g., low vascularization) could significantly improve wound outcomes and avoid prolonged care settings that could lead to secondary complications (i.e., PUs) and impact unit readiness and return-to-duty rates. The deployment of new medical capabilities to the operational theater, however, is challenging. Novel and simpler to implement technologies with enhanced capabilities are still needed for early treatment of military wounds.

Many military injuries are amenable to tissue transplant and regenerative medicine treatments. Gene/cell therapies have emerged as promising alternative strategies for wound healing, especially in the presence of detrimental comorbidities (e.g., infection, diabetes, EB). Current approaches to gene/cell therapies, however, face multiple hurdles, including safety concerns due to heavy reliance on viral vectors, tumorigenesis, and immunogenicity. Moreover, problems encountered in Combat Casualty Care include the remote and often austere environment in which the battle occurs, limited access to necessary supplies and equipment to implement care, and the need to transport casualties expeditiously across dangerous terrain to a safe environment for further recovery. As such, current gene/cell therapy-based wound management strategies are unlikely to serve as a first line of defense in the military theater.

There remains a need for improved compositions and methods for therapeutic, prophylactic, or diagnostic applications in wound healing that overcome the aforementioned deficiencies.

### SUMMARY

Described herein are electrospun core-shell fibers that include (i) a central core that is electrically conductive having an exterior surface, wherein the core comprises a first polymer and an electroconductive material; (ii) a shell adjacent to the exterior surface of the core, the shell comprising a second polymer; and (iii) one or more bioactive agents in the shell. In one aspect, the fibers are electrospun fibers. Additionally, described herein are methods for making and using the core-shell fibers.

Other systems, methods, features, and advantages of compositions and methods described herein will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 is a schematic diagram illustrating aspects of one exemplary nanofibrous dressing with tissue nano-transfection (TNT) capabilities for controlled delivery of gene/cell therapies.

FIGS. 2A-2C demonstrate that proper fiber orientation improves cell migration rates. FIG. 2A depicts microscopy image at t=0 h for aligned (left column), counter aligned (middle column), and randomly oriented (right column) fibers. FIG. 2B depicts microscopy image at t=8 h for aligned (left column), counter aligned (middle column), and randomly oriented (right column) fibers. FIG. 2C is a graph of the cell migration rate (μm/hr) for the aligned, counter aligned, and randomly oriented fibers respectively.

FIG. 3 is a series of photographs demonstrating the wound healing over 28 days for control (top) and nanofiber (bottom) wound dressings.

FIGS. 4A-4C demonstrate the existence of nanochannels on the fiber surface (FIG. 4A) which can be used to apply TNT. FIG. 4B demonstrates mouse skin tissue TNT′d with a GFP plasmid using a nanofiber sheet/dressing format (right) compared to a control skin was TNT′d with mock/empty plasmids (left). FIG. 4C demonstrates that nanofiber dressings can also be micronized into a powder nanowhisker format for easy application/injection.

FIGS. 5A-5C depict schematics of exemplary core-shell fibers according to various aspects of the disclosure including either a solid biodegradable shell (FIG. 5B and left figure in FIG. 5A) or a nanostructured or nanoporous shell (FIG. 5C and right figure in FIG. 5A).

FIG. 6 shows a prototype transfection device according to one aspect of the present disclosure.

FIG. 7 shows a comparison of GFP expression in transfected skin versus control skin according to one aspect of the present disclosure.

FIG. 8 shows immunofluorescence of skin after transfection with pmaxGFP compared to a control.

FIG. 9 shows immunofluorescence of skin after transfection with genes that induce reprogramming into islet-like tissue in the skin compared to a control.

FIG. 10 shows immunofluorescence of skin after transfection with genes that induce reprogramming into neuron-like tissue in the skin compared to a control.

FIG. 11A is a brightfield image of fiber powders (dark areas) according to the present disclosure loaded with a DDK flag/reporter after injection into the flank of mice. After 3 days, transfection was conducted and skin was biopsied and visualized. FIG. 11B shows a fluorescence image of the transfected, biopsied skin of FIG. 11A, indicating DDK expression.

FIG. 12A shows is a brightfield image of control fiber powders (dark areas). After 3 days, skin was biopsied and visualized. FIG. 12B shows a fluorescence image of the biopsied skin of FIG. 12A, showing no DDK expression in the control group.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting. The skilled artisan will recognize many variants and adaptations of the embodiments described herein. These variants and adaptations are intended to be included in the teachings of this disclosure.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant specification should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Although any methods and materials similar or equivalent to those described herein can also be used in the practice or testing of the present disclosure, the preferred methods and materials are now described. Functions or constructions well-known in the art may not be described in detail for brevity and/or clarity. Embodiments of the present disclosure will employ, unless otherwise indicated, techniques of nanotechnology, organic chemistry, material science and engineering, pharmacy, pharmacology, medicine, wound care, and the like, which are within the skill of the art. Such techniques are explained fully in the literature.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

In some instances, units may be used herein that are non-metric or non-SI units. Such units may be, for instance, in U.S. Customary Measures, e.g., as set forth by the National Institute of Standards and Technology, Department of Commerce, United States of America in publications such as NIST HB 44, NIST HB 133, NIST SP 811, NIST SP 1038, NBS Miscellaneous Publication 214, and the like. The units in U.S. Customary Measures are understood to include equivalent dimensions in metric and other units (e.g., a dimension disclosed as “1 inch” is intended to mean an equivalent dimension of “2.5 cm”; a unit disclosed as “1 pcf” is intended to mean an equivalent dimension of 0.157 kN/m³; or a unit disclosed 100° F. is intended to mean an equivalent dimension of 37.8° C.; and the like) as understood by a person of ordinary skill in the art.

### Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

The term “wound,” as used herein, refers to physical disruption of the continuity or integrity of tissue structure. “Wound healing” refers to the restoration of tissue integrity. It will be understood that this can refer to a partial or a full restoration of tissue integrity. Treatment of a wound thus refers to the promotion, improvement, progression, acceleration, or otherwise advancement of one or more stages or processes associated with the wound healing process.

“Parenteral administration”, as used herein, means administration by any method other than through the digestive tract or non-invasive topical or regional routes. For example, parenteral administration may include administration to a patient intravenously, intradermally, intraperitoneally, intrapleurally, intratracheally, intramuscularly, subcutaneously, subjunctivally, by injection, and by infusion.

“Topical administration”, as used herein, means the non-invasive administration to the skin, orifices, or mucosa. Topical administrations can be administered locally, i.e. they are capable of providing a local effect in the region of application without systemic exposure. Topical formulations can provide systemic effect via adsorption into the blood stream of the individual. Topical administration can include, but is not limited to, cutaneous and transdermal administration, buccal administration, intranasal administration, intravaginal administration, intravesical administration, ophthalmic administration, and rectal administration.

In some aspects, the compounds, compositions, and methods disclosed herein can be utilized with or on a subject in need of treatment, which can also be referred to as “in need thereof.” As used herein, the phrase “in need thereof means that the subject has been identified as having a need for the particular method or treatment and that the treatment has been given to the subject for that particular purpose.

The term “subject” as used herein includes, but is not limited to, humans, nonhuman vertebrates, and animals such as wild, domestic, and farm animals. Preferably, the term “subject” refers to mammals. More preferably, the term “subject” refers to humans.

A “therapeutically effective amount” or “effective amount” of a composition is a predetermined amount calculated to achieve the desired effect (e.g., amount of bioactive agent to treat a wound). The core-shell fibers described herein are effective over a wide dosage range. It will be understood that the effective amount administered will be determined by the physician, veterinarian, or other medical professional in the light of the relevant circumstances including the condition to be treated, the choice of compound to be administered, and the chosen route of administration, and therefore the dosage ranges described herein are not intended to limit the scope of the disclosure in any way.

The terms “treat,” “treated,” or “treating” as used herein refer to both therapeutic treatment and prophylactic or preventative measures, wherein the object is to prevent or slow down (lessen) or entirely reverse (eradicate) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; remission (whether partial or total), whether detectable or undetectable, or enhancement or improvement of the condition, disorder, or disease; and eradication of the condition, disorder, or disease. Treatment includes eliciting a clinically significant response without excessive levels of side effects. Treatment also includes prolonging survival as compared to expected survival if not receiving treatment.

Core-Shell Fibers and Methods of Making

A variety of core-shell fibers are provided herein as well as fiber clusters, textiles, meshes, mats, and various formulations thereof that can be used for the efficient delivery of bioactive agents. In some aspects, core-shell fibers are provided that include (i) a central core that is electrically conductive having an exterior surface, wherein the core comprises a first polymer and an electroconductive material; (ii) a shell adjacent to the exterior surface of the core, the shell comprising a second polymer.

Polymers

A variety of different polymers can be used to produce the core and shell of the fibers described herein. In one aspect, the first polymer in the core can be the same polymers as the second polymer in the shell. In another aspect, the first polymer and the second polymer are different polymers. In other aspects, the first and second polymer are biocompatible.

In one aspect, the first and second polymer can be a synthetic or semi-synthetic polymers such as, without limitation, polyethylene terephthalate, a polyester, a polymethylmethacrylate, polyacrylonitrile, a silicone, a polyurethane, a polycarbonate, a polyether ketone ketone, a polyether ether ketone, a polyether imide, a polyamide, a polystyrene, a polyether sulfone, a polysulfone, a polycaprolactone (PCL), a polylactic acid (PLA), a polyglycolic acid (PGA), a polylactide-co-glycolide copolymer (PLGA), a polyglycerol sebacic, a polydiol citrate, a polyhydroxy butyrate, a polyether amide, a polydiaxanone, or any combination thereof.

In another aspect, the first and second polymer can be a natural polymer such as, for example, fibronectin, collagen, gelatin, hyaluronic acid, chitosan, or combinations thereof. It may be understood that the first polymer and/or second polymer may also include a combination of synthetic polymers and naturally occurring polymers in any combination or compositional ratio.

In another aspect, the first and second polymer can be a biocompatible polymer selected from the group consisting of polyalkylene glycols such as poly(ethylene glycol) and poly(propylene glycol); aliphatic polyesters based on hydroxyalkanoic acids, such as poly(lactic acid), poly(glycolic acid), poly(e-caprolactone) and related copolymers; poly(methyl methacrylate); poly(ethylene vinyl acetate); poly(2-hydroxyethyl methacrylate); polyvinylpyrrolidone; copolymers thereof; and blends thereof.

In certain aspects, the first polymer and second polymer can be formulated as solutions prior to forming the core-shell fiber. For example, the first polymer and second polymer can independently be formulated with one or more solvents to produce compositions or solutions suitable for electrospinning fibers. In one aspect, the solvent can be acetone, dimethylformamide, dimethylsulfoxide, N-methylpyrrolidone, acetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof.

When formulated with solvents, the amount of the first polymer and second polymer can vary. In one aspect, the first polymer is from 1 wt % to 50 wt % of the composition, or about 1 wt %, 5 wt %, 10 wt %, 15 wt %, 20 wt %, 25 wt %, 30 wt %, 35 wt %, 40 wt %, 45 wt %, or 50 wt %, where any value can be a lower and upper endpoint of a range (e.g., 5 wt % to 25 wt %). In another aspect, the second polymer is from 1 wt % to 50 wt % of the composition, or about 1 wt %, 5 wt %, 10 wt %, 15 wt %, 20 wt %, 25 wt %, 30 wt %, 35 wt %, 40 wt %, 45 wt %, or 50 wt %, where any value can be a lower and upper endpoint of a range (e.g., 5 wt % to 25 wt %).

Electroconductive Materials

The shell of the core-fiber includes an electroconductive material in addition to the first polymer. An electroconductive material is any material capable of conducting electricity. In one aspect, the electroconductive material is an electroconductive polymer. Examples of electroconductive polymers include, but are not limited to, polyaniline, poly(pyrrole)s, oxidized polyacetylenes, poly(fluorene)s, polyphenylenes, polypyrenes, polyazulenes, polynaphthalenes, poly(p-phenylene vinylene), polycarbazoles, polyindoles, polyazepines, poly(thiophene)s, poly(3,4-ethylenedioxythiophene), poly(p-phenylene sulfide), poly(naphthalene vinylene)s, poly(3,4-ethylenedioxythiophene)-poly(styrenesulfonate), poly(3,4-ethylenedioxythiophene)-block-poly(ethylene glycol), or any combination thereof. In one aspect, the electroconductive polymer can include one polymer or two or more polymers.

In one aspect, the electroconductive material is an electroconductive metal. Examples of electroconductive metals include, but are not limited to, tantalum, gold, niobium, silver, copper, aluminum, iron, zinc, molybdenum, lithium, nickel, palladium, platinum, tungsten, tin, rhodium, Iridium, or any combination thereof. In one aspect, the electroconductive metal can include one metal or two or more metals. In one aspect, the electroconductive metal can be nanoparticles having an average diameter of about 1 nm, 10 nm, 50 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, or 1,000 nm, where any value can be a lower and upper endpoint of a range (e.g., 100 nm to 500 nm).

In one aspect, the electroconductive material can include a combination of one or more electroconductive polymers with one or more electroconductive metals. For example, the electroconductive material can be a combination of polyaniline and tantalum particles.

When formulated with solvents, the amount of the first polymer and electroconductive material can vary. In one aspect, the first polymer is from 1 wt % to 50 wt % of the composition, or about 1 wt %, 5 wt %, 10 wt %, 15 wt %, 20 wt %, 25 wt %, 30 wt %, 35 wt %, 40 wt %, 45 wt %, or 50 wt %, where any value can be a lower and upper endpoint of a range (e.g., 5 wt % to 25 wt %) and the electroconductive polymer is from 1 wt % to 50 wt % of the composition, or about 1 wt %, 5 wt %, 10 wt %, 15 wt %, 20 wt %, 25 wt %, 30 wt %, 35 wt %, 40 wt %, 45 wt %, or 50 wt %, where any value can be a lower and upper endpoint of a range (e.g., 5 wt % to 25 wt %). In another aspect, the first polymer is from 1 wt % to 50 wt % of the composition, or about 1 wt %, 5 wt %, 10 wt %, 15 wt %, 20 wt %, 25 wt %, 30 wt %, 35 wt %, 40 wt %, 45 wt %, or 50 wt %, where any value can be a lower and upper endpoint of a range (e.g., 5 wt % to 25 wt %) and the electroconductive metal is from 1 wt % to 90 wt % of the composition, or about 1 wt %, 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, or 90 wt %, where any value can be a lower and upper endpoint of a range (e.g., 5 wt % to 25 wt %).

In one aspect, the weight ratio of the first polymer to the electroconductive polymer in the core-shell fiber is from 2:1 to 1:2, or is 2:1, 1.5:1, 1;1, 1:1.5, or 1:2, where any value can be a lower and upper endpoint of a range (e.g., 1.5:1 to 1:1.5). In another aspect, the weight ratio of the first polymer to the electroconductive metal in the core-shell fiber is from 1:10 to 1:1, or is 1:10, 1:9, 1:8, 1:7, 1:6, 1:5, 1:4, 1:3, 1:2, or 1:1, where any value can be a lower and upper endpoint of a range (e.g., 1:10 to 1:7).

Electrospinning

The core-shell fibers described herein are electrospun fibers. Electrospinning is a method of spinning a polymer fiber or polymer nanofiber from a polymer solution by applying a high DC voltage potential between the polymer solution (or polymer injection system containing the polymer solution) and a receiving surface for the electrospun polymer nanofibers. The voltage potential may include voltages less than or equal to about 15 kV. The polymer may be ejected by a polymer injection system at a flow rate of less than or equal to about 5 mL/h. As the polymer solution travels from the polymer injection system toward the receiving surface, it may be elongated into sub-micron diameter electrospun polymer nanofibers, typically in the range of about 0.1 μm to about 10 μm. Some non-limiting examples of electrospun polymer nanofiber diameters may include about 0.1 μm, about 0.2 μm, about 0.5 μm, about 1 μm, about 2 μm, about 5 μm, about 10 μm, about 20 μm, or ranges between any two of these values (including endpoints). [0043] A polymer injection system may include any system configured to eject some amount of a polymer solution into an atmosphere to permit a flow of the polymer solution from the injection system to the receiving surface. In some non-limiting examples, the injection system may deliver a continuous stream of a polymer solution to be formed into a polymer nanofiber. In alternative examples, the injection system may be configured to deliver intermittent streams of a polymer to be formed into multiple polymer nanofibers. In one embodiment, the injection system may include a syringe under manual or automated control. In another embodiment, the injection system may include multiple syringes under individual or combined manual or automated control. In some examples, a multi-syringe injection system may include multiple syringes, each syringe containing the same polymer solution. In alternative examples, a multi-syringe injection system may include multiple syringes, each syringe containing a different polymer solution.

The receiving surface may move with respect to the polymer injection system, or the polymer injection system may move with respect to the receiving surface. In some embodiments, the receiving surface may move with respect to the polymer injection system under manual control. In other embodiments, the surface may move with respect to the polymer system under automated control. In such embodiments, the receiving surface may be in contact with or mounted upon a support structure that may be moved using one or more motors or motion control systems. In some non-limiting examples, the surface may be a roughly cylindrical surface configured to rotate about a long axis of the surface. In some other non-limiting examples, the surface may be a flat surface that rotates about an axis approximately coaxial with the polymer fiber ejected by the polymer injection system. In yet some other non-limiting examples, the surface may be translated in one or more of a vertical direction and a horizontal direction with respect to the polymer nanofiber ejected by the polymer injection system. It may be further recognized that the receiving surface of the polymer nanofiber may move in any one direction or combination of directions with respect to the polymer nanofiber ejected by the polymer injection system. The pattern of the electrospun polymer nanofiber deposited on the receiving surface may depend upon the one or more motions of the receiving surface with respect to the polymer injection system. In one non-limiting example, a roughly cylindrical receiving surface, having a rotation rate about its long axis that is faster than a translation rate along a linear axis, may result in a roughly helical deposition of an electrospun polymer fiber forming windings about the receiving surface. In an alternative example, a receiving surface having a translation rate along a linear axis that is faster than a rotation rate about a rotational axis, may result in a roughly linear deposition of an electrospun polymer fiber along a liner extent of the receiving surface.

In some embodiments, the receiving surface may be coated with a non-stick material, such as, for example, aluminum foil, a stainless steel coating, polyteirafluoroethylene, or a combination thereof, before the application of the electrospun polymer nanofibers. The receiving surface, such as a mandrel, may be fabricated from aluminum, stainless steel, polytetrafluoroethylene, or a combination thereof to provide a nonstick surface on which the electrospun nanofibers may be deposited. In other embodiments, the receiving surface may be coated with a simulated cartilage or other supportive tissue. In some non-limiting examples, the receiving surface may be composed of a planar surface, a circular surface, an irregular surface, and a roughly cylindrical surface. One embodiment of a roughly cylindrical surface may be a mandrel. A mandrel may take the form of a simple cylinder, or may have more complex geometries. In some non-limiting examples, the mandrel may take the form of a hollow bodily tissue or organ. In some non-limiting examples, the mandrel may be matched to a subject's specific anatomy.

In one aspect, the core-shell fiber is produced by electrospinning a concentric composition comprising an inner first composition comprising the first polymer and an electroconductive polymer and a second outer composition comprising the second polymer. In one aspect, a higher gauge needle can be inserted into a lower gauge needle. A syringe with the first polymer and electroconductive material will be injected into the higher gauge needle concurrently while the second polymer is injected into the lower gauge needle during electrospinning to produce the core-shell fiber. Upon electrospinning, the shell layer is adjacent to (i.e., intimate contact with) the core such that a shell is formed around the entire core. The Examples provide non-limiting methods for making the core-shell fibers described herein.

The thickness of the shell layer can vary depending upon the application of the core-shell fibers. For example, where it is desirable to deliver larger amounts of bioactive agent, the thickness of the shell can be increased to hold more of the agent. In one aspect, the shell has a thickness of about 10 nm to about 20 μm, or 10 nm, 50 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm, where any value can be a lower and upper endpoint of a range (e.g., 800 nm to 15 μm).

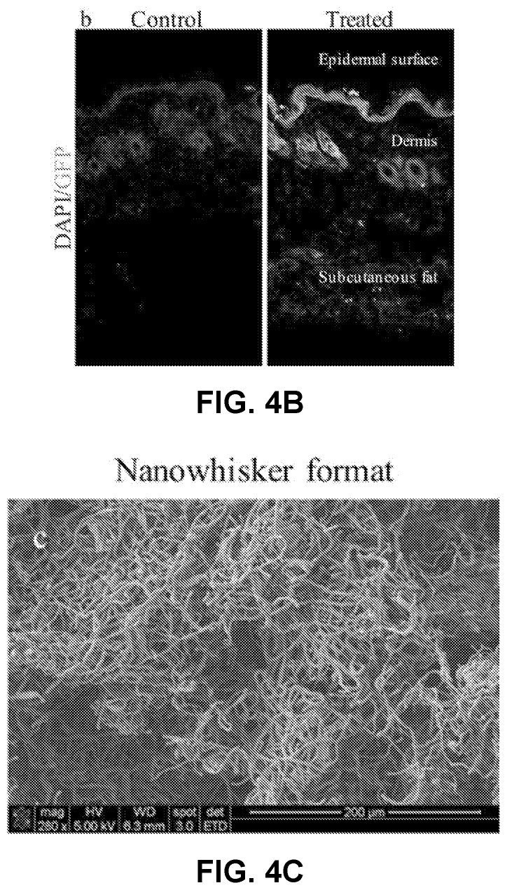

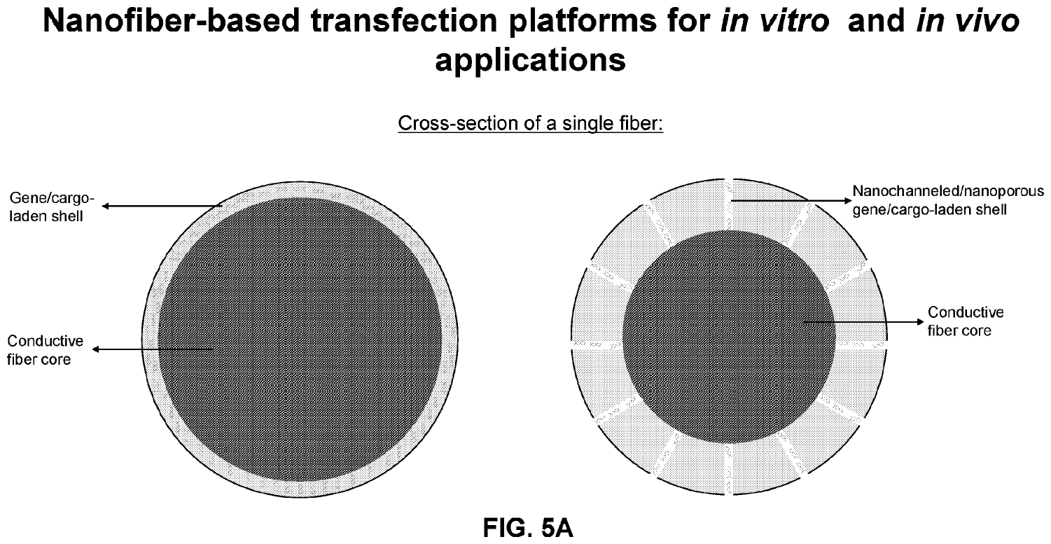

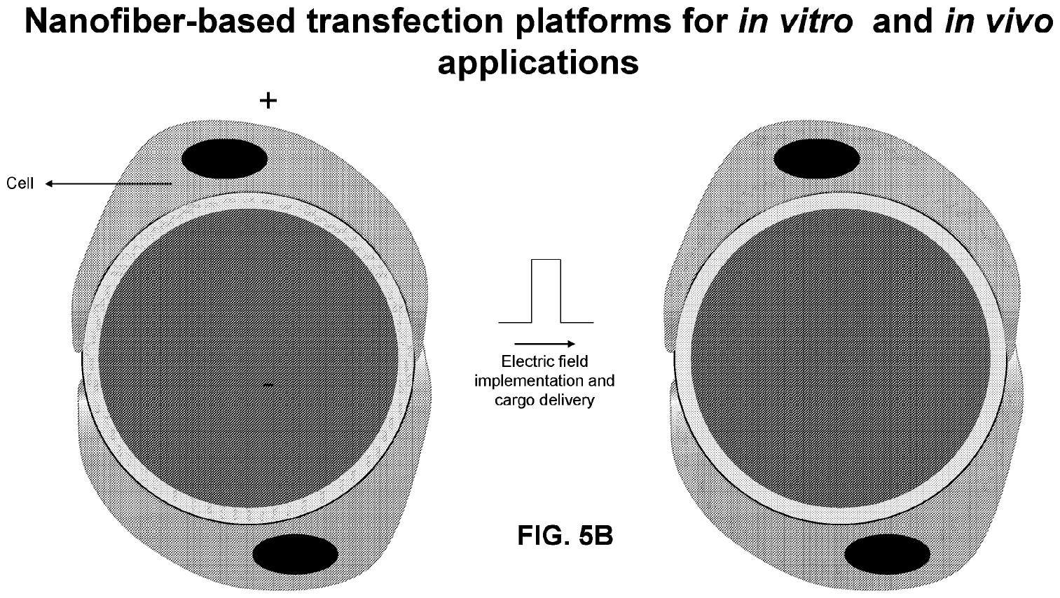

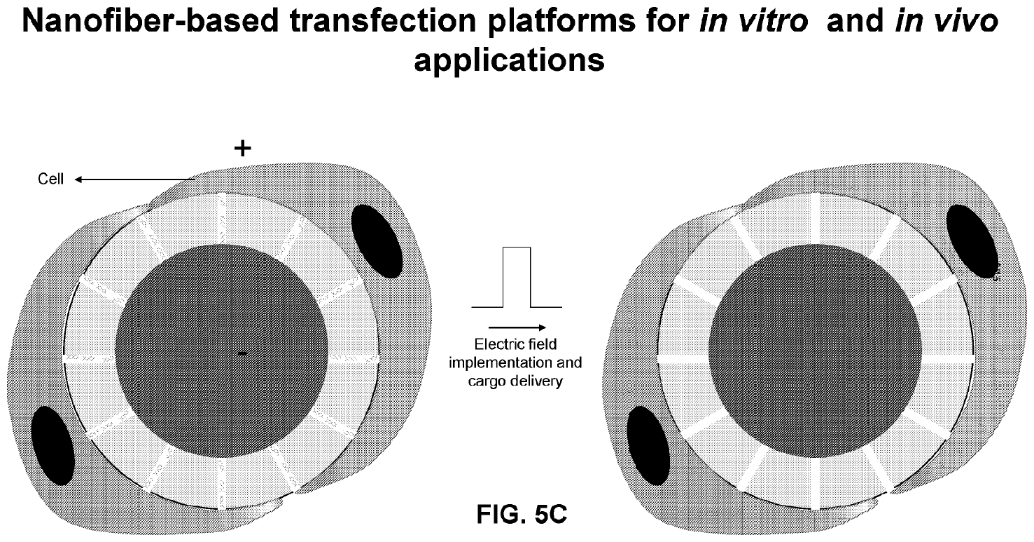

In certain aspects, the shell comprises a plurality of nanochannels. FIG. 4A depicts nanochannels in the fiber. In one aspect, the nanochannels have an average diameter of about 10 nm to about 1,000 nm, or 10 nm, 50 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, or 1,000 nm, where any value can be a lower and upper endpoint of a range (e.g., 200 nm to 750 nm). The nanochannels can be made by a variety of techniques. In one aspect, the second polymer can be formulated in a solvent that is immiscible with water. Upon electrospinning in a humid environment and subsequent evaporation of the solvent, nanochannels are produced.

In one aspect, the core-shell fibers described herein can be produced by electrospinning a plurality of fiber fragments. As used herein, the term “fragment” refers to a portion of a particular fiber. In some aspects, a fragment can have an average length of about 1 μm to about 1,000 μm, and an average diameter of about 0.1 μm to about 10 μm. Some non-limiting examples of average fragment lengths may include an average length of about 1 μm, an average length of about 5 μm, an average length of about 10 μm, an average length of about 20 μm, an average length of about 30 μm, an average length of about 40 μm, an average length of about 50 μm, an average length of about 75 μm, an average length of about 90 μm, an average length of about 95 μm, an average length of about 100 μm, an average length of about 105 μm, an average length of about 125 μm, an average length of about 150 μm, an average length of about 200 μm, an average length of about 300 μm, an average length of about 400 μm, an average length of about 500 μm, an average length of about 600 μm, an average length of about 700 μm, an average length of about 800 μm, an average length of about 900 μm, an average length of about 1000 μm, or ranges between any two of these values (including endpoints).

Some non-limiting examples of average fragment diameters may include an average diameter of about 0.1 μm, an average diameter of about 0.5 μm, an average diameter of about 1 μm, an average diameter of about 2 μm, an average diameter of about 3 μm, an average diameter of about 4 μm, an average diameter of about 5 μm, an average diameter of about 6 μm, an average diameter of about 7 μm, an average diameter of about 8 μm, an average diameter of about 9 μm, an average diameter of about 10 μm, or ranges between any two of these values (including endpoints). When combined with a carrier medium, the resulting mixture may include from about 1 fragment per mm³ to about 100,000 fragments per mm³. Some non-limiting examples of mixture densities may include about 2 fragments per mm³, about 100 fragments per mm³, about 1,000 fragments per mm³, about 2,000 fragments per mm³, about 5,000 fragments per mm³, about 10,000 fragments per mm³, about 20,000 fragments per mm³, about 30,000 fragments per mm³, about 40,000 fragments per mm³, about 50,000 fragments per mm³, about 60,000 fragments per mm³, about 70,000 fragments per mm³, about 80,000 fragments per mm³, about 90,000 fragments per mm³, about 100,000 fragments per mm³, or ranges between any two of these values (including endpoints).

As used herein, the term “cluster” refers to an aggregate of fiber fragments, or a linear or curved three-dimensional group of fiber fragments. Clusters may have a range of shapes. Non-limiting examples of cluster shapes may include spherical, globular, ellipsoidal, and flattened cylinder shapes. Clusters may have, independently, an average length of about 1 μm to about 1000 μm, an average width of about 1 μm to about 1000 μm, and an average height of about 1 μm to about 1000 μm. It may be appreciated that any cluster dimension, such as length, width, or height, is independent of any other cluster dimension. Some non-limiting examples of average cluster dimensions include an average dimension (length, width, height, or other measurement) of about 1 μm, an average dimension of about 5 μm, an average dimension of about 10 μm, an average dimension of about 20 μm, an average dimension of about 30 μm, an average dimension of about 40 μm, an average dimension of about 50 μm, an average dimension of about 75 μm, an average dimension of about 90 μm, an average dimension of about 95 μm, an average dimension of about 100 μm, an average dimension of about 105 μm, an average dimension of about 110 μm, an average dimension of about 150 μm, an average dimension of about 200 μm, an average dimension of about 300 μm, an average dimension of about 400 μm, an average dimension of about 500 μm, an average dimension of about 600 μm, an average dimension of about 700 μm, an average dimension of about 800 μm, an average dimension of about 900 μm, an average dimension of about 1000 μm, or ranges between any two of these values (including endpoints), or independent combinations of any of these ranges of dimensions. Clusters may include an average number of about 2 to about 1000 fiber fragments. Some non-limiting examples of average numbers of fiber fragments per cluster include an average of about 2 fiber fragments per cluster, an average of about 5 fiber fragments per cluster, an average of about 10 fiber fragments per cluster, an average of about 20 fiber fragments per cluster, an average of about 30 fiber fragments per cluster, an average of about 40 fiber fragments per cluster, an average of about 50 fiber fragments per cluster, an average of about 60 fiber fragments per cluster, an average of about 70 fiber fragments per cluster, an average of about 80 fiber fragments per cluster, an average of about 90 fiber fragments per cluster, an average of about 100 fiber fragments per cluster, an average of about 110 fiber fragments per cluster, an average of about 200 fiber fragments per cluster, an average of about 300 fiber fragments per cluster, an average of about 400 fiber fragments per cluster, an average of about 500 fiber fragments per cluster, an average of about 600 fiber fragments per cluster, an average of about 700 fiber fragments per cluster, an average of about 800 fiber fragments per cluster, an average of about 900 fiber fragments per cluster, an average of about 1000 fiber fragments per cluster, or ranges between any two of these values (including endpoints). In some embodiments, a composition may contain a plurality of clusters.

In some aspect, the core-shell fibers described herein can be used to produce a textile. The term “textile” is defined herein as a spun, woven, or otherwise fabricated material comprising the core-shell fibers described herein. The textile can include meshes, mats, and the like. In the case when the core-shell fiber is electrospun to produce a mesh or mat, the fibers can be aligned or substantially aligned (i.e., greater than 95% aligned). In other aspects, the fibers can be randomly aligned.

In some aspects, the core-shell fibers may be wound about a mandrel, as threads are wound around a bobbin. In some aspects, the core-shell fibers may be deposited, in an essentially parallel manner, along a linear dimension of a mandrel or other surface form. In some embodiments, winding the textile may use electrospinning techniques. The term “pore size” is thus defined herein as being the diameter of introduced pores, pockets, voids, holes, spaces, etc. introduced in an unmeshed structure such as a block polymer, polymer sheet, or formed polymer scaffold, and is specifically distinguished from “mesh size” as disclosed herein. As used herein, the term “mesh size” is the number of openings in a textile per linear measure. For example, if the textile has 1200 openings per linear millimeter, the textile is defined 1200 mesh (e.g., sufficient to allow a 12 micron red blood cell to pass), which is easily convertible between imperial and metric units. A mesh size may be determined based on the number of fibers having a specified average diameter and an average opening size between adjacent fibers along a specified linear dimension. Thus, a textile composed of 10 μm average diameter fibers having 10 μm average diameter openings between adjacent fibers may have about 50 total openings along a 1 mm length and may therefore be defined as a 50 mesh textile.

In some aspects, the core-shell fiber is spun over a mandrel so as to form a textile roll or tube. In some embodiments, the thickness of the textile roll or tube may be regulated by changing the number of rotations of the mandrel over time while the textile roll or tube collects the fiber. In certain other embodiments, the biocompatible textile has a mesh size of about 1 opening per mm to about 20 openings per mm. Some non-limiting examples of mesh sizes may include about 1 opening per mm, about 2 openings per mm, about 4 openings per mm, about 6 openings per mm, about 8 openings per mm, about 10 openings per mm, about 15 openings per mm, about 20 openings per mm, or ranges between any two of these values (including endpoints). In some embodiments, the mesh size of the spun textile may be about 20 openings per mm to about 500 openings per mm. Some non-limiting examples of mesh sizes may include about 20 openings per mm, about 40 openings per mm, about 60 openings per mm, about 80 openings per mm, about 100 openings per mm, about 200 openings per mm, about 300 openings per mm, about 400 openings per mm, about 500 openings per mm, or ranges between any two of these values (including endpoints). In other embodiments, the mesh size may be about 500 openings per mm to about 1000 openings per mm. Some non-limiting examples of mesh sizes may include about 500 openings per mm, about 600 openings per mm, about 700 openings per mm, about 800 openings per mm, about 1000 openings per mm, or ranges between any two of these values (including endpoints). In some embodiments, the mesh size of the textile may be regulated by changing the speed and direction by which the fiber is deposited onto the mandrel, such as, by example, moving the position and direction in which the thread is spun onto the textile roll or tube.

Bioactive Agents

The core-shell fibers can be loaded with any bioactive agent (e.g., therapeutic, prophylactic, or diagnostic agents) compatible with the shell, i.e. with any agent that can be loaded into the polymers of the shell. Preferred agents that can be loaded include genes useful for therapeutic, prophylactic, or diagnostic effect.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As mentioned previously, the agents can include genes and other nucleic acids. The agents can include an aptamer such as RNA, DNA or an artificial nucleic acid. The agent can be an anionic proteins, protein analogues, or nucleic acids.

The terms “nucleic acid,” “polynucleotide,” and “oligonucleotide” are used interchangeably to refer to a deoxyribonucleotide or ribonucleotide polymer, in linear or circular conformation, and in either single- or double-stranded form. These terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general and unless otherwise specified, an analogue of a particular nucleotide has the same base-pairing specificity; i.e., an analogue of A will base-pair with T. The term “nucleic acid” is a term of art that refers to a string of at least two base-sugar-phosphate monomeric units. Nucleotides are the monomeric units of nucleic acid polymers. The term includes deoxyribonucleic acid (DNA) and ribonucleic acid (RNA) in the form of a messenger RNA, antisense, plasmid DNA, parts of a plasmid DNA or genetic material derived from a virus. Antisense is a polynucleotide that interferes with the function of DNA and/or RNA. The term nucleic acids refers to a string of at least two base-sugar-phosphate combinations. Natural nucleic acids have a phosphate backbone, artificial nucleic acids may contain other types of backbones, but contain the same bases. The term also includes PNAs (peptide nucleic acids), phosphorothioates, and other variants of the phosphate backbone of native nucleic acids.

A “functional fragment” of a protein, polypeptide or nucleic acid is a protein, polypeptide or nucleic acid whose sequence is not identical to the full-length protein, polypeptide or nucleic acid, yet retains at least one function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA binding function of a polypeptide can be determined, for example, by filter-binding, electrophoretic mobility shift, or immunoprecipitation assays. DNA cleavage can be assayed by gel electrophoresis. The ability of a protein to interact with another protein can be determined, for example, by co-immunoprecipitation, two-hybrid assays or complementation, e.g., genetic or biochemical. See, for example, Fields et al. (1989) Nature 340:245-246; U.S. Pat. No. 5,585,245 and PCT WO 98/44350.

In one aspect, the bioactive agent can include one or more of a nucleic acid, a peptide, a polypeptide, a small molecule, a vaccine, vesicles isolated from cells that have been reprogrammed, and a combination thereof. The one or more therapeutic, prophylactic, or diagnostic agents can include one or more of genes such as LL37, laminin/collagen VII, and VEGF/EGF. The one or more bioactive agents can include RNA, DNA or an artificial nucleic acid. The one or more bioactive agents can include anionic proteins, protein analogues, or nucleic acids. The one or more therapeutic, prophylactic, or diagnostic agents can encode proteins selected from the group consisting of ETV2, FOXC2, and FLI1.

The bioactive agents can be loaded into/onto the core-shell fibers in several different methods. In one aspect, the bioactive agent can be added into solution of the second polymer before electrospinning. In another aspect, the bioactive agent can be adsorbed onto the fiber surface after electrospinning by soaking the fibers in a solution of the cargo. In another aspect, the bioactive agent can be embedded into the fibers via subcritical/supercritical carbon dioxide.

In one aspect, the bioactive agent is present only in the shell and not the core of the core-shell fibers. In certain aspects, when the shell comprises nanochannels, the bioactive agent is present in the nanochannels.

In certain aspects, when the core-shell fibers are electrospun into a mesh or mat, the bioactive agent can be adsorbed at different concentrations at different locations on the mat or mesh to produce a concentration gradient. Depending upon the application of the mesh or mat, different amounts of bioactive agent can be added to the mesh or mat. In other aspects, different bioactive agents can be applied to the mesh or mat at different locations. The nature and amount of the bioactive agent used and applied to the mesh or mat can vary depending upon the application.

Pharmaceutical Formulations, Medicaments, and Bandages

Parenteral Formulations

The fibers can be formulated for parenteral delivery, such as injection or infusion, in the form of a solution or suspension. The formulation can be administered via any route, such as, the blood stream or directly to the organ or tissue to be treated.

In one aspects, the core-shell fibers described herein can be ground pulverized into a produce nanowhiskers (FIG. 4C) using techniques known in the art. For example, a mesh or mat produced by the electrospun core-shell fibers can be grounded into powders that can subsequently formulated in an aqueous solution suitable for injection

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the core-shell particles can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the fibers and/or the therapeutic, prophylactic, or diagnostic agent.

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the nanoparticles in the required amount in the appropriate solvent or dispersion medium with one or more of the excipients listed above, as required, followed by filtered sterilization. Generally, dispersions are prepared by incorporating the various sterilized nanoparticles into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the nanoparticle plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration are preferably in the form of a sterile aqueous solution or suspension of the fibers. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol.

In some instances, the formulation is distributed or packaged in a liquid form. Alternatively, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for parenteral administration may also contain one or more tonicity agents to adjust the isotonic range of the formulation. Suitable tonicity agents are well known in the art and some examples include glycerin, mannitol, sorbitol, sodium chloride, and other electrolytes.

Solutions, suspensions, or emulsions for parenteral administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for parenteral administration may also contain one or more excipients known art, such as dispersing agents, wetting agents, and suspending agents.

Topical Formulations

In other aspects, the core-shell fibers described herein can be formulated for topical administration. In one aspect, the core-shell fibers can exist as a pulverized materials (i.e., nanowhiskers) that can be applied directly to a subject (e.g., sprinkled on a wound).

In another aspect, the powder form of the core-shell fibers can be formulated with a topical carrier. Suitable dosage forms for topical administration include creams, ointments, salves, sprays, balms, gels, lotions, emulsions, liquids, and transdermal patches. The formulation may be formulated for transmucosal, transepithelial, transendothelial, or transdermal administration. The compositions contain one or more chemical penetration enhancers, membrane permeability agents, membrane transport agents, emollients, surfactants, stabilizers, and combination thereof.

In some embodiments, the core-shell fibers can be administered as a liquid formulation, such as a solution or suspension, a semi-solid formulation, such as a lotion or ointment, or a solid formulation. In some embodiments, the fibers are formulated as liquids, including solutions and suspensions, such as a semi-solid formulation, such as ointment or lotion for topical application to the skin, to the mucosa.

The formulation may contain one or more excipients, such as emollients, surfactants, emulsifiers, penetration enhancers, and the like.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Suitable classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of the nanoparticles in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, it may be desirable to provide continuous delivery of one or more fibers to a patient in need thereof. For topical applications, repeated application can be done or a patch can be used to provide continuous administration of the fibers over an extended period of time.

In certain aspects, the core-shell fibers can be applied to or integrated with a wound dressing, gauge, or bandage for wound care. In one aspect, the core-shell fibers can be applied directly to the wound dressing, gauge, or bandage as a powder or solution. In another aspect, when the core-shell fibers are in a mesh or mat, the mesh or mat can function as the bandage and be applied directly to a wound.

Implants

In other aspects, the core-shell fibers described herein can be used to produce an implant. As used herein, the term “implant” may refer to any structure that may be introduced for a permanent or semi-permanent period of time into a body. An implant may have the shape and size of a native organ or tissue that it may serve to replace. Alternatively, an implant may have a shape not related to a specific bodily organ or tissue. In yet another embodiment, an implant may have a shape of an entire bodily organ or tissue, or only a portion thereof. In still another example, an implant may be shaped like a portion of a bodily organ or tissue, or may simply comprise a patch of material. In still another example, a composition for implantation may be flexible and not rigidly shaped, and may mold or form to the area to which it is applied. The implant may be designed for introduction into a body of an animal, including a human.

Methods of Delivering Bioactive Agents

The core-shell fibers described herein are useful in administering bioactive agents to a subject. In one aspect, the method involves

administering or applying to the subject the core-shell fibers or fiber clusters described herein; and

applying an electric field to the fibers or fiber cluster.

The application of the electric field to the core-shell fibers promote, localized, focused release of the of the bioactive agent to a specific region of the subject. In general, fibers loaded with bioactive agents do not have controlled release of the bioactive agent. The core-shell fibers described herein address this need. It is also possible to control the amount of bioactive agent that is released from the core-shell fibers. For example, by varying the voltage and the duration of the electrical field that is applied, it is possible to vary the amount of bioactive agent that is released. The electrical field that is applied to the core-shell fibers can be continuous or pulsed. In one aspect, the electric field has a voltage of from about 100 volts to about 500 volts, or about 100 volts, 150 volts, 200 volts, 250 volts, 300 volts, 350 volts, 400 volts, 450 volts, or 500 volts, where any value can be a lower and upper endpoint of a range (e.g., 200 volts to 300 volts).

In one aspect, the electric field can be applied to the core-shell fibers with the use of electrodes. For example, when the core-shell fibers are configured into a mat or mesh, a negative electrode is attached to the mesh or mat and a positive electrode is gently attached to the subject (e.g., the skin). In another aspect, when the core-shell fibers are administered to the subject via injection, the positive and negative electrodes are gently attached around the injection site. The core-shell fibers can be applied at any location on the subject. In the case of a wound, the core-shell fibers can be applied directly to the wound or near the wound.

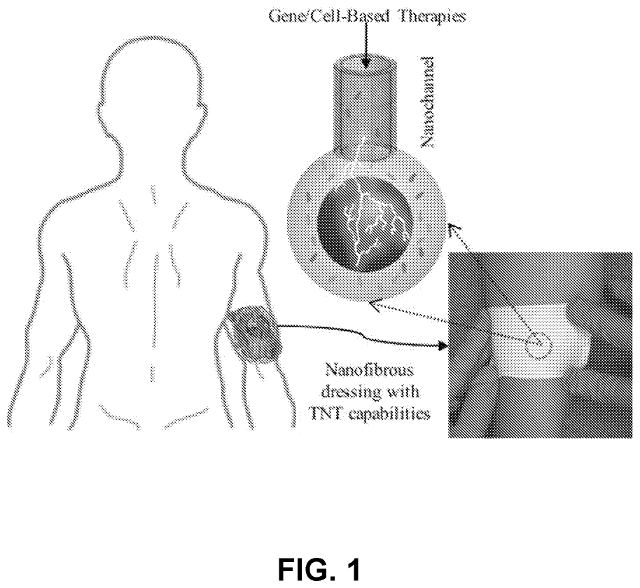

In various aspects, the core-shell fibers described herein prevent and/or interrupt wound infections, induce or improving wound healing in subjects with impaired wound healing (e.g. diabetes), enhance wound healing, and provide sustained release of drugs in tissue repair. In various aspects, the core-shell fibers described herein are capable of actively and controllably delivering gene/cell and/or electroceutical therapies to injured tissues via Tissue Nano-Transfection (TNT) (FIG. 1).

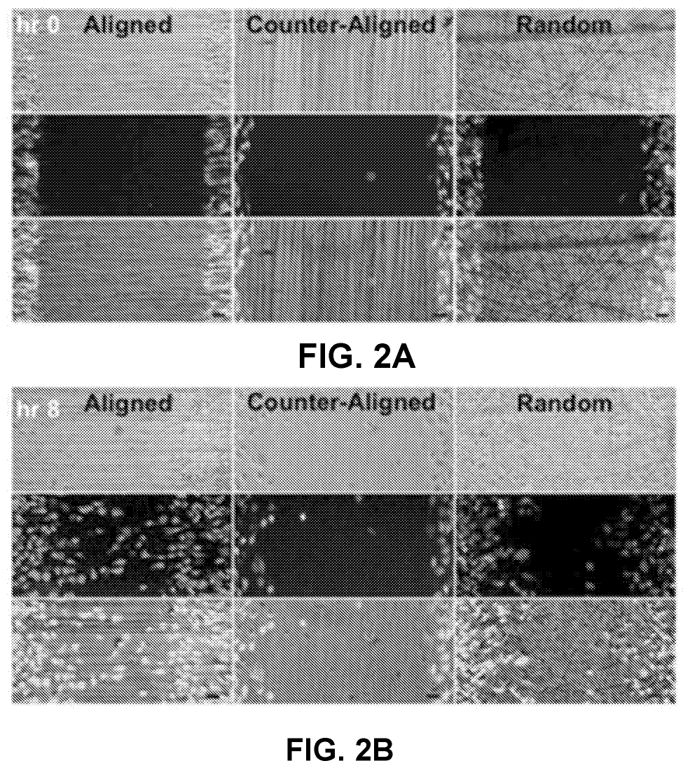

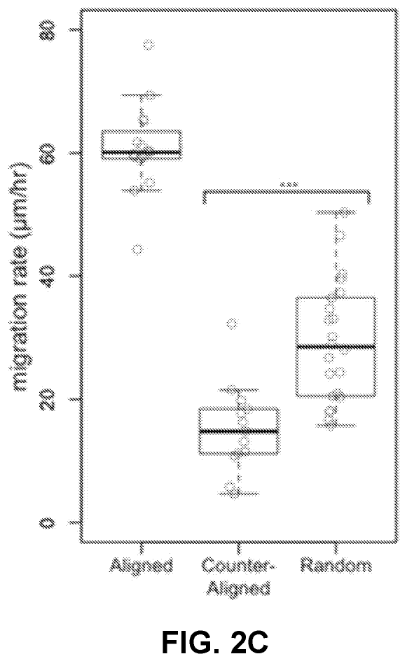

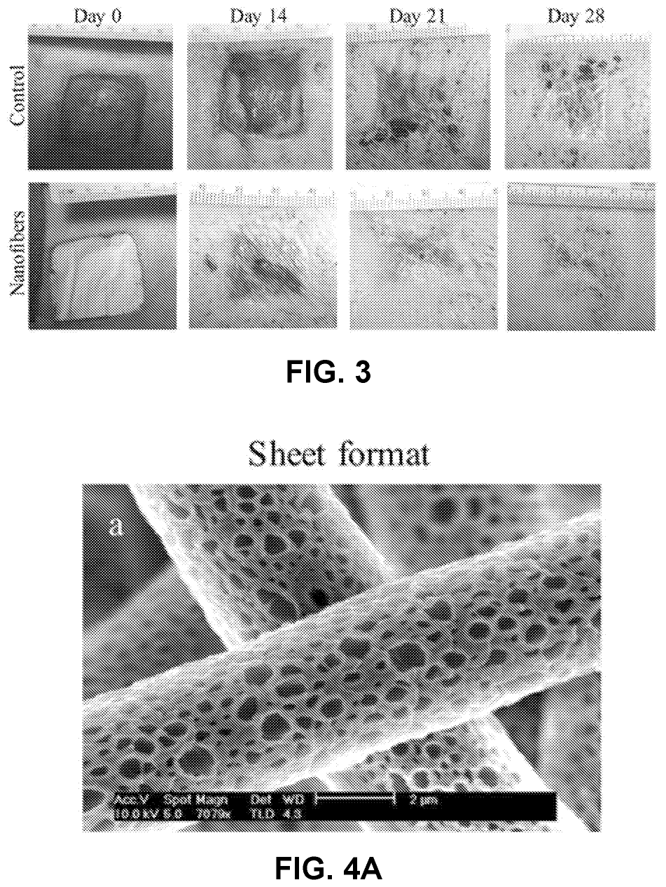

The electrospun core-shell fibers described herein can serve as an ideal tissue replacement scaffold for a variety of tissues. As described herein, electrospun nanofiber technology can be designed for the efficient delivery of bioactive agents to a subject wound healing and has the potential to enable rapid and flexible responses to mass casualty events. In addition to being able to tailor fiber chemistry to modulate tissue-fiber interactions and biodegradation dynamics, fibers can be electrospun in an aligned manner to potentiate wound closure rates (FIGS. 2A-2C). Porcine studies indicate that, compared to standard gauze dressings, nanofiber-dressed wounds healed approximately 25% faster and with less scarring (FIG. 3).

There has been a growing push to incorporate more active elements to improve healing, including gene delivery mechanisms. Prior art methods for delivering genes to wounds, however, are fraught with caveats, including heavy reliance on viral vectors. Safety concerns hamper clinical implementation, and although adeno-associated viruses (AVV) are less pathogenic, AVV-host interactions and immunity are still a major concern. Prior art non-viral approaches (e.g., bulk electroporation, synthetic nanocarriers), on the other hand, tend to induce toxicity and inflammation, and exhibit low transfection efficiency. The core-shell fibers described herein address these shortcomings.

Tissue nano-transfection (TNT) is an emergent technology capable of highly efficient non-viral delivery of gene and/or cell therapies to pristine or injured tissues. Existing TNT approaches, however, are currently driven by nanomachined silicon (Si) hardware, which is costly and fragile, and is thus not suitable for treating large/uneven wounds, or for implantation/long-term treatment. As such, while promising, current TNT hardware is unlikely to support point-of-care/conflict intervention as wound dressing. Incorporating TNT capabilities into dressing platforms, as described herein enables simultaneous delivery of powerful gene/cell therapies. The core-shell fibers described herein enables the development of next-generation of TNT-based delivery of bioactive agents such as genes.

In one aspect, the core-shell fibers described herein are useful in treating a wound in a subject in need thereof. The wound may be acute or chronic. Chronic wounds, including pressure sores, venous leg ulcers and diabetic foot ulcers, can simply be described as wounds that fail to heal. Whilst the exact molecular pathogenesis of chronic wounds is not fully understood, it is acknowledged to be multi-factorial. As the normal responses of resident and migratory cells during acute injury become impaired, these wounds are characterised by a prolonged inflammatory response, defective wound extracellular matrix (ECM) remodelling and a failure of re-epithelialisation. The methods can include applying a wound dressing, gauge, or bandage containing the core-shell fibers described herein. The methods can include applying a wound dressing, gauge, or bandage described herein to the wound. The methods can also include applying a pharmaceutical formulation described herein to the wound, e.g. applying an ointment or cream described herein to the wound.

The wound may be any internal wound, e.g. where the external structural integrity of the skin is maintained, such as in bruising or internal ulceration, or external wounds, particularly cutaneous wounds, and consequently the tissue may be any internal or external bodily tissue. In one embodiment the tissue is skin (such as human skin), i.e. the wound is a cutaneous wound, such as a dermal or epidermal wound. In some aspects, the pharmaceutical formulation is applied via enteral administration, e.g. via intradermal or subcutaneous injection.

Wounds can be classified in one of two general categories, partial thickness wounds or full thickness wounds. A partial thickness wound is limited to the epidermis and superficial dermis with no damage to the dermal blood vessels. A full thickness wound involves disruption of the dermis and extends to deeper tissue layers, involving disruption of the dermal blood vessels. The healing of the partial thickness wound occurs by simple regeneration of epithelial tissue. Wound healing in full thickness wounds is more complex. In some aspects, the wounds are cutaneous wounds which may be either partial thickness or full thickness wounds.

The wounds include cuts and lacerations, surgical incisions or wounds, punctures, grazes, scratches, compression wounds, abrasions, friction wounds (e.g. nappy rash, friction blisters), decubitus ulcers (e.g. pressure or bed sores); thermal effect wounds (burns from cold and heat sources, either directly or through conduction, convection, or radiation, and electrical sources), chemical wounds (e.g. acid or alkali burns) or pathogenic infections (e.g. viral, bacterial or fungal) including open or intact boils, skin eruptions, blemishes and acne, ulcers, chronic wounds, (including diabetic-associated wounds such as lower leg and foot ulcers, venous leg ulcers and pressure sores), skin graft/transplant donor and recipient sites, immune response conditions, e.g. psoriasis and eczema, stomach or intestinal ulcers, oral wounds, including a ulcers of the mouth, damaged cartilage or bone, amputation wounds and corneal lesions.

Suitable effective amounts (dosage) and dosing regimens can be determined by the attending physician and may depend on the particular tissue type and wound being treated, the nature and severity of the wound, i.e. whether partial or full thickness, chronic or acute, as well as the general age, and health of the subject.

The formulation of compositions and dressings contemplated herein is well known to those skilled in the art, see for example, Remington's Pharmaceutical Sciences, 18^{th} Edition, Mack Publishing, 1990. Compositions may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. The carrier for compositions contemplated by the present invention must be pharmaceutically acceptable in the sense of being compatible with the other ingredients of the composition and not injurious to the subject. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

In another aspect, the core-shell fibers described herein can be useful for the delivery genes in gene therapy. In a further aspect, gene delivery by the core-shell fibers described herein can be useful for replacing a mutated gene that causes disease with a healthy copy of the gene such as, for example, with cystic fibrosis, Duchenne muscular dystrophy, hemophilia, certain forms of glaucoma, retinitis pigmentosa, genetic amyotrophic lateral sclerosis, Rett syndrome, tyrosinemia type 1, spinal muscular atrophy, alpha-1 antitrypsin deficiency, severe combined immunodeficiency, adenine deaminase deficiency, familial hypercholesterolemia, or the like. In another aspect, gene delivery can be useful for inactivating a mutated gene that is functioning improperly such as, for example, with Huntington's disease, glycogen and lysosomal storage disorders, sickle cell disease, thalassemias, epidermolysis bullosa, and the like. In still another aspect, a new gene can be introduced into the body to help fight a disease such as, for example, HIV, hepatitis B, herpes simplex virus, human papilloma virus, or another viral infection, a gastrointestinal bacterial infection, or the like. In some aspects, gene delivery can be useful for T-cell immunotherapy and/or other ways of boosting a subject's immune response. In one aspect, gene delivery by the core-shell fibers described herein can be useful in the treatment of cancers including, but not limited to, brain, lung, breast, pancreatic, liver, colorectal, prostate, bladder, head and neck, skin, ovarian, and/or renal cancer. In a further aspect, delivery of genes by the core-shell fibers described herein to cancerous tissue or surrounding tissue can cause cell death or slow the growth of the cancer, or can make other treatments such as, for example, chemotherapy or radiation more effective. In another aspect, a drug may have systemic side effects in active form but be relatively harmless in prodrug form. Further in this aspect, gene products can be delivered to cells where the drug's activity is needed, wherein the gene products are configured to convert the prodrug to the active drug form to treat a disease such as, for example, cancer.

In other aspects, the core-shell fibers described herein can be used to repair damaged tissue in a subject. In other aspects, the core-shell fibers described herein can be used to repair damaged nerve tissue. Regeneration and repair in the nervous system is a process by which damaged tissue undergoes regrowth or renewal, leading to eventual restoration of nervous system function. This process happens more readily with axons, synapses, neurons and glia in the peripheral nervous system. In another aspect, the core-shell fibers described herein can be used to repair damage to muscles, ligaments, blood vessels, and readily accessible portions of the eye (e.g., sclera, cornea, conjunctiva), and/or portions that can be reached via injection such as, for example, the lens, pupil, retina.

It will be understood that the methods may also be practiced in conjunction with the use of other supplementary biologically or physiologically active agents. Thus, the methods and compositions described herein may be used in conjunction with other biologically or physiologically active agents such as antiviral agents, antibacterial agents, antifungal agents, vitamins, such as A, C, D and E and their esters, and/or additional wound healing agents, including a growth factors and cytokines, such as those described herein. These additional agents may be formulated into a composition or dressing together with fibers described herein.

The following listing of exemplary aspects supports and is supported by the disclosure provided herein.

Aspect 1. An electrospun core-shell fiber comprising:
(i) a central core that is electrically conductive having an exterior surface, wherein the core comprises a first polymer and an electroconductive material;
(ii) a shell adjacent to the exterior surface of the core, the shell comprising a second polymer; and
(iii) one or more bioactive agents in the shell.
Aspect 2. The fiber of Aspect 1, wherein the electroconductive material comprises an electroconductive polymer, an electroconductive metal, or a combination thereof.
Aspect 3. The fiber of Aspect 1 or 2, wherein the electroconductive polymer comprises polyaniline, polyaniline, a poly(pyrrole), an oxidized polyacetylene, a poly(fluorene), a polyphenylenes, a polypyrene, a polyazulene, a polynaphthalene, a poly(p-phenylene vinylene), a polycarbazole, a polyindoles, a polyazepine, a poly(thiophene), a poly(3,4-ethylenedioxythiophene), a poly(p-phenylene sulfide), a poly(naphthalene vinylene), a poly(3,4-ethylenedioxythiophene)-poly(styrenesulfonate), a poly(3,4-ethylenedioxythiophene)-block-poly(ethylene glycol), or any combination thereof
Aspect 4. The fiber of any one of Aspects 1-3, wherein the weight ratio of the first polymer to the electroconductive polymer is from 2:1 to 1:2.
Aspect 5. The fiber of any one of Aspects 1-4, wherein the electroconductive metal comprises tantalum, gold, niobium, silver, copper, aluminum, iron, zinc, molybdenum, lithium, nickel, palladium, platinum, tungsten, tin, rhodium, Iridium, or any combination thereof.
Aspect 6. The fiber of any one of Aspects 1-5, wherein the electroconductive metal comprises a plurality of metal nanoparticles.
Aspect 7. The fiber of any one of Aspects 1-6, wherein the weight ratio of the first polymer to the electroconductive metal is from 1:10 to 1:1.
Aspect 8. The fiber of any one of Aspects 1-7, wherein the electroconductive material comprises a combination of one or more electroconductive polymers and one or more electroconductive metals
Aspect 9. The fiber of any one of Aspects 1-8, wherein the first polymer is biocompatible.
Aspect 10. The fiber of any one of Aspects 1-9, wherein the first polymer comprises a synthetic polymer comprising polyethylene terephthalate, a polyester, a polymethylmethacrylate, polyacrylonitrile, a silicone, a polyurethane, a polycarbonate, a polyether ketone ketone, a polyether ether ketone, a polyether imide, a polyamide, a polystyrene, a polyether sulfone, a polysulfone, a polycaprolactone (PCL), a polylactic acid (PLA), a polyglycolic acid (PGA), a polylactide-co-glycolide copolymer (PLGA), a polyglycerol sebacic, a polydiol citrate, a polyhydroxy butyrate, a polyether amide, a polydiaxanone, or any combination thereof.
Aspect 11. The fiber of any one of Aspects 1-10, wherein the first polymer comprises a natural polymer comprising fibronectin, collagen, gelatin, hyaluronic acid, chitosan, or any combination thereof.
Aspect 12. The fiber of one of Aspects 1-10, wherein the first polymer comprises poly(e-caprolactone), polylactic acid (PLA), polyglycolic acid (PGA), polylactide-co-glycolide copolymer (PLGA), or any combination thereof.
Aspect 13. The fiber of any one of Aspects 1-12, wherein the core has an average diameter of about 100 nm to about 20 μm.
Aspect 14. The fiber of any one of Aspects 1-13, wherein the second polymer is biocompatible.
Aspect 15. The fiber of any one of Aspects 1-14, wherein the second polymer comprises a synthetic polymer comprising polyethylene terephthalate, a polyester, a polymethylmethacrylate, polyacrylonitrile, a silicone, a polyurethane, a polycarbonate, a polyether ketone ketone, a polyether ether ketone, a polyether imide, a polyamide, a polystyrene, a polyether sulfone, a polysulfone, a polycaprolactone (PCL), a polylactic acid (PLA), a polyglycolic acid (PGA), a polylactide-co-glycolide copolymer (PLGA), a polyglycerol sebacic, a polydiol citrate, a polyhydroxy butyrate, a polyether amide, a polydiaxanone, or any combination thereof.
Aspect 16. The fiber of any one of Aspects 1-14, wherein the second polymer comprises a natural polymer comprising fibronectin, collagen, gelatin, hyaluronic acid, chitosan, or any combination thereof.
Aspect 17. The fiber of one of Aspects 1-14, wherein the second polymer comprises poly(e-caprolactone), polylactic acid (PLA), polyglycolic acid (PGA), polylactide-co-glycolide copolymer (PLGA), or any combination thereof.
Aspect 18. The fiber of any one of Aspects 1-17, wherein the first polymer and the second polymer are the same polymer.
Aspect 19. The fiber of any one of Aspects 1-18, wherein the first polymer and the second polymer are the different polymers.
Aspect 20. The fiber of any one of Aspects 1-19, wherein the shell has a thickness of about 10 nm to about 20 μm.
Aspect 21. The fiber of any one Aspects 1-20, wherein the shell comprises a plurality of nanochannels.
Aspect 22. The fiber of any one Aspects 1-21, wherein the nanochannels have an average diameter of about 10 nm to about 1,000 nm.
Aspect 23. The fiber of any one Aspects 1-22, wherein the one or more bioactive agents comprise one or more of a nucleic acid, a peptide, a polypeptide, a small molecule, a vaccine, vesicles isolated from cells that have been reprogrammed, and any combination thereof.
Aspect 24. The fiber of any one Aspects 1-22, wherein the one or more bioactive agents comprise one or more of genes such as LL37, laminin/collagen VII, and VEGF/EGF.
Aspect 25. The fiber of any one Aspects 1-24, wherein the fibers are continuous.
Aspect 26. A bioactive core-shell fiber produced by the method comprising contacting a core-shell fiber with a bioactive agent, wherein the core-shell fiber comprises:
(i) a central core that is electrically conductive having an exterior surface, wherein the core comprises a first polymer and an electroconductive material; and
(ii) a shell adjacent to the exterior surface of the core, the shell comprising a second polymer.
Aspect 27. The fiber of Aspect 26, wherein the core-shell fiber is produced by electrospinning a concentric composition comprising an inner first composition comprising the first polymer and an electroconductive polymer and a second outer composition comprising the second polymer.
Aspect 28. A fiber cluster comprising a plurality of fibers of any one of Aspects 1-27.
Aspect 29. The fiber cluster of Aspect 28, wherein the plurality of fibers are aligned or substantially aligned.
Aspect 30. The fiber cluster of Aspect 28, wherein the plurality of fibers are randomly aligned.
Aspect 31. The fiber cluster of any one Aspects 28-30, wherein fiber cluster has a fragment size of about 50 μm to about 500 μm.
Aspect 32. The fiber cluster of any one Aspects 28-31, wherein the plurality of fibers comprise a plurality of long fibers and a plurality of short fibers.
Aspect 33. A pharmaceutical formulation comprising a therapeutically effective amount of the fibers of any one of Aspects 1-27 or the fiber clusters of any one of Aspects 28-32 and a suitable carrier.
Aspect 34. The pharmaceutical formulation of Aspect 33, wherein the formulation comprises a topical formulation.
Aspect 35. The pharmaceutical formulation of Aspect 33 or 34, wherein the formulation is in the form of a cream, a balm, an ointment, a spray, a lotion, an emulsion, a gel, a salve, or a transdermal patch.
Aspect 36. The pharmaceutical formulation of Aspect 33, wherein the formulation comprises a parenteral formulation.
Aspect 37. The pharmaceutical formulation of Aspect 36, wherein the formulation is suitable for one or both of subcutaneous injection and intradermal injection.
Aspect 38. A mat or mesh comprising a therapeutically effective amount of the fibers of any one of Aspects 1-27 or the fiber clusters of any one of Aspects 28-32.
Aspect 39. The mat or mesh of Aspect 39, wherein the mat or mesh comprises the bioactive agent having different concentrations at different locations on the mat or mesh.
Aspect 40. A wound dressing, gauge, or bandage comprising a therapeutically effective amount of the fibers of any one of Aspects 1-27 or the fiber clusters of any one of Aspects 28-32.
Aspect 41. A textile or implant comprising a therapeutically effective amount of the fibers of any one of Aspects 1-27 or the fiber clusters of any one of Aspects 28-32.
Aspect 42. A method for delivering a bioactive agent to a subject, the method comprising
(i) administering or applying to the subject the fibers of any one of Aspects 1-27 or the fiber clusters of any one of Aspects 28-32; and
(ii) applying an electric field to the fibers or fiber cluster.
   Aspect 43. The method of Aspect 42, wherein the method comprises administering to the subject the pharmaceutical formulation of any one of Aspects 33-37.
   Aspect 44. The method of Aspect 42, wherein the method comprises applying to the subject the mesh or mat of Aspect 38 or 39.
   Aspect 45. The method of any one of Aspects 42-44, wherein the electric field has a voltage of from about 100 volts to about 500 volts.
   Aspect 46. The method of any one of Aspects 42-45, wherein the electric field is applied continuously or is applied as a pulse.
   Aspect 47. The method of any one of Aspects 42-46, wherein the subject has a wound, wherein the fibers or fiber cluster are applied at or near the wound.
   Aspect 48. The method of Aspect 47, wherein the wound comprises an acute wound or a chronic wound.
   Aspect 49. The method of Aspect 47, wherein the wound comprises a skin wound.
   Aspect 50. The method of Aspect 49, wherein the skin wound comprises a partial thickness wound or full thickness wound.
   Aspect 51. The method of Aspect 50, wherein the wound comprises a cut, laceration, surgical incision, puncture, graze, scratch, compression wound, abrasion, friction wound, decubitus ulcer; thermal effect wound, chemical wounds, wound caused by a pathogenic infections, ulcer, a diabetic-associated wound, skin graft/transplant donor and recipient sites, immune response condition, stomach or intestinal ulcer, oral wound, damaged cartilage or bone, amputation wound, or corneal lesion.
   Aspect 52. The method of Aspect 47, wherein the wound comprises nerve tissue damage.
   Aspect 53. The method of Aspect 47, wherein the wound comprises damage to a muscle, ligament, blood vessel, or eye.
   Aspect 54. The method of any one of Aspects 42-46, wherein the subject is in need of gene delivery.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Prophetic Example 1. Development of Nanofibrous Dressings with TNT Ability

Nanofiber dressings with different designs are manufactured to incorporate electrically conductive elements. Conductive elements include one or both of conductive polymer and tantalum nanoparticles. The designs include examples with and without nanochannels to impart TNT capabilities. Thus, in addition to fulfilling structural functions (e.g., scaffolding, absorption), these dressings will actively and controllably deliver genes to the wound non-virally (i.e., by nanochannel electroporation). Transfection studies in diabetic and healthy mice are conducted to identify optimum dressing designs for wound healing studies. The toxicity of these dressings in skin (1^{ry} exposure site) and alveolar cells (potential 2^{ry} exposure site for nanowhiskers) is also evaluated using standard assays.

Polymer nanofibers are electrospun into mats using a custom designed electrospinning setup. For the blended configuration with conducting polymer (CP_{b}), polymeric solutions will be prepared by mixing 6 wt % PLGA8218 (Purac) and 5 wt % polyaniline (PANi) (Sigma) in hexafluoroisopropanol (HFIP) (Oakwood) at RT. For the blended configuration with tantalum nanoparticles (NP_{b}), 50 wt % of tantalum nanoparticles (100 nm) will be added to the 6 wt % PLGA solution before electrospinning. The polymer solutions will then be placed in a 60 cc plastic syringe and electrospun at 5 mL/h with +15 kV on the needle, −4 kV on the collection mandrel, and a 20 cm tip-to-collector distance. For the core-shell configuration, fibers will be spun with a conductive core made out of either PANi (CP_{cs}) or tantalum nanoparticles (NP_{cs}), and an insulating shell made out of PLGA. Briefly, fibers will be prepared by using a 22-gauge hypodermic needle inserted through a 16-gauge hypodermic T-junction (Small Parts) to create two concentric blunt needle openings. A Swagelok stainless steel union will hold the needles in place and ensure the ends of the needles are flush with each other. One syringe (BD Luer-Lok tip) will be filled with the polymer solution for the core (PLGA+PANi or PLGA+tantalum nanoparticles), connected to the 22-gauge needle and set to a 5 mL/h flow rate using a syringe pump. Another syringe will be connected via an extension to the T-junction, filled with the shell material (6 wt % PLGA solution) and set to a flow rate of 0.5 mL/h with another syringe pump. Electrospinning will then be performed as described above. All the meshes will be spun to a thickness of approximately 250 μm and then used as-is, or mechanically milled into nanowhiskers. Morphological, chemical and electrical characterization will be simultaneously conducted in terms of scanning electron microscopy (SEM), energy dispersive spectroscopy (EDS), and in situ conductivity measurements at 0-21 days post-manufacturing. Additional characterization will be conducted by transmission electron microscopy (TEM) and dynamic light scattering (DLS) as detailed below.

Nanowhisker formulations of CP_{b}, NP_{b}, CP_{cs}, and NP_{cs} will be suspended into sterile 1:80 phosphate buffered saline (PBS) solution at a stock concentration of 2 mg/mL. Size and aspect ratio will be determined by blotting a droplet of the suspension on TEM grids and imaging. Surface charge will be determined by DLS zeta potential analysis. Toxicity analyses will be conducted using in vitro alveolar and skin cell Transwell® (TW) co-culture models. The lung model will consist of a 90:10 T1A alveolar type-I to H441 alveolar type-II co-culture interfaced with microvascular endothelial cells lining the basolateral side of the porous membrane. While, the skin model will consist of HaCaT cells co-cultured with fibroblasts interfaced with microvascular endothelial cells. Epithelial cells will be cultured for two days under submerged conditions to reach confluence, then the TW will be turned over and the endothelial cells added. After two hours of adherence, the TW will be placed back up-right and allowed to culture for an additional two days prior to removing the apical medium creating an air-liquid interface (ALI) culture. After three days of ALI culture the basolateral medium will be replaced, transepithelial electrical resistance (TEER) values recorded (CelIZScope), and the units placed into a VitroCell Cloud aerosol deposition system. The VitroCell Cloud-generated aerosol will be optimized prior to exposure to determine dose conditions relevant to the wound healing procedure. TW cultures will be exposed to the aerosol under physiological conditions, placed back into the incubator, and control samples exposed to the suspension solvent under the same exposure conditions. TEER recordings will be collected every 8 h up to 48 h post-exposure, when the basolateral medium will be collected, the apical surface washed with Hank's balanced salt solution, and the wash eluent collected. Some samples will be trypsinized, apical and basolateral surfaces separately, collected, concentrated, and aliquoted into three vials. One vial will be used for apoptosis/necrosis assessment via flow cytometry. Another vial will be assayed with H2DCF-DA reactive oxygen species probe and evaluated using a flow cytometry. The last vial will be assayed for alveolar type-I or II markers, the endothelial cells stained for CD31 or ICAM (indicating activation), and the fibroblast stained for alpha-smooth muscle actin and subsequently analyzed using flow cytometry to quantify cellular fractions. One sample per group will be fixed in paraformaldehyde, permeabilized, and stained for the nuclei (DAPI), f-actin, and tight-junction formation. The basolateral supernatant will be evaluated using an ELISA kit for cytokine secretion, and the apical lung models for surfactant production. Results will be compared to null and positive control (LPS) conditions.

Transfection studies will be first conducted on pristine skin of type 2 diabetic (T2D) db/db mice (8-12-week-old). These mice exhibit many of the hallmarks seen in T2D humans, such as metabolic syndrome and associated complications (e.g., impaired wound healing). healthy/heterozygous mice (db/+) will be used for comparison purposes. All procedures will be conducted in mice anesthetized with isoflurane. Prior to transfection, the mouse dorsum will be depilated. The exposed skin will be exfoliated with 0.25% trypsin to remove the layers of dead/enucleated cells and expose the stratum spinosum. Sheets (precut into 8-10 mm diameter discs) and nanowhiskers of CP_{b}, NP_{b}, CP_{cs}, and NP_{cs} will be pre-incubated with the gene plasmid (LL37, laminin/collagen VII, and VEGF/EGF) solutions (0.1 mg/μl) at RT for 24 h in a vacuum desiccator to foment plasmid incorporation within the nanochannels. All plasmids will be purchased from Origene/Addgene, and diluted in PBS at 0.05 μg/μl. For sheet-based transfection, the nanofiber discs will be conformally applied directly on the skin surface, and connected to the negative lead of a BTX power supply. The positive lead will be inserted subdermally, adjacent or juxtaposed to the fiber disc. This will then be followed by the implementation of a pulsed electric field across both leads (250V, 10 ms pulses, 10 pulses). A single fiber disc will be used to sequentially transfect at least 5 different locations on the mouse dorsum. For the nanowhisker version, ˜50 μl of the whisker/plasmid solution will be first intradermally injected on the dorsum of the mice, and the injection site will be clamped between the positive and negative leads of the power supply, followed by implementation of a pulsed electric field as described above. In this case, transfections will be conducted immediately after, or 1-3 days post-injection to allow for tissue integration/ingrowth to occur. Nanowhiskers will be injected in at least 5 different locations of the dorsum, and each location will be pulsed either 1, 2, 3, 4 or 5 times every 24 h. Mock plasmids (i.e., empty vectors/plasmids with the same backbone) will be used as control. Following the last transfection, the mice will be collected at days 1, 3, 7, 14 and 21, and skin biopsies will be processed for gene expression (i.e., qRT-PCR) and protein immunostaining (with antibodies against LL37, laminin/collagen VII, VEGF/EGF, CD31 and CD105) analyses. Skin cytoarchitecture will be evaluated by hematoxylin/eosin (H&E) staining. Cell apoptosis/necrosis in response to transfection will be evaluated via immunostaining for caspase-3, and counterstaining for ethidium homodimer. Prior to collection, potential changes in functional outcomes, including permeability and perfusion, will be evaluated via transepidermal water loss (TEWL) and laser speckle (LSI) imaging, respectively.

We expect to successfully develop and evaluate blended and core-shell fiber configurations with different compositions. Core-shell fibers isolate the conductive element (core) from the surrounding tissue, which may lead to improved viability post-transfection compared to blended fibers. However, blended fibers may result in electric field maximization at the fiber surface and thus improved electrophoretic motility of the plasmid DNA and transfection outcomes. Likewise, although tantalum nanoparticles are more conductive than PANi, and as such are expected to lead to stronger electric fields and improved transfection, this may come at the expense of tissue viability. Repeated transfection experiments will allow us to establish metrics for how many transfections can be conducted with a single platform and still maintain successful gene transfer. The results from this aim (i.e., viability and gene/protein expression level post-transfection) will be used to select an optimum fiber configuration (blended vs. core-shell) and composition (PANi− vs. tantalum nanoparticles).

### Prophetic Example 2. To Evaluate the Extent to which Nanofibrous Dressings with TNT Ability Improve Wound Healing

Using the designs from Prophetic Example 1, we will evaluate the extent to which nanofiber-based TNT of pro-healing genes (VEGF, EGF and LL37) improves wound outcomes in db/db and db/+mice. We will evaluate this on wounds dressed or sprinkled with TNT-capable nanofiber sheets or nanowhiskers, respectively.

A full-thickness splint model will be used for these studies. TNT-capable dressings (sheets and nanowhiskers) of optimum performance will be manufactured and gene-impregnated as described above. Three different types of dressing will be tested: (1) LL37-loaded, (2) VEGF/EGF-loaded (1:1 VEGF:EGF molar ratio), and (3) LL37/VEGF/EGF-loaded (1:1:1 LL37:VEGF:EGF molar ratio). LL37 has been shown to also play a positive role in wound healing beyond bactericidal⁴. Mice will be anesthetized using isoflurane, and the dorsum will be depilated and cleaned using betadine. A sterile 6-mm punch biopsy tool will be used to make full-thickness paired wounds (4/mouse) equidistant from the midline and adjacent to the four limbs. A donut-shaped splint with an 8 mm inner diameter will be created from a 0.5 mm-thick silicone sheet (Grace Bio-Laboratories) and placed such that the wound is centered. To fix the splint to the skin, an immediate-bonding adhesive will be used followed by interrupted 6-0 nylon sutures (Ethicon) to maintain position. The nanofiber dressings will be applied (sheets, 2 wounds/mouse) or sprinkled (nanowhiskers, 2 wounds/mouse) on the exposed wound bed. To evaluate the combined effect of topical (i.e., wound bed level) and deep gene delivery on wound healing, 50% of the wound beds (2/mouse) will be pre-injected with nanowhiskers prior to applying the sheet or sprinkling the nanowhiskers. Control groups will include: (1) dressed wounds TNT′d with mock plasmids, (2) dressed wounds with no electric field implementation, and (3) wounds dressed with standard gauze.

Once the dressing is placed/sprinkled, we will apply a pulsed electric field as described above. In this case, however, a split electrode will be used for the positive lead, which will be subdermally inserted, parallel and adjacent to the wound edge. Slight pressure will be exerted with the negative lead to guarantee conformal contact between the dressing material and the wound bed, and to minimize the orthogonal gap between the negative and split/positive electrodes. Mice/wounds will be transfected either once, immediately after dressing, or up to two to three times with a 7-day gap between transfections.

Digital images of the wounds will be taken on days 1, 3, 7, 14, 21 and 28 post-wounding. Wound area measurement will be done by digital planimetry in Image-J. Wound perfusion and skin permeability will be monitored at the same time points by LSI and TEWL measurements. The quality of healing will be determined by histopathological measurements from tissue biopsied at days 7, 14, 21 and 28 post-wounding. Tissue sections will be stained with H&E, and antibodies against LL37, VEGF, EGF, CD31 and CD105.

The results will demonstrate the extent to which nanofibrous dressing-driven TNT of pro-healing genes improves wound outcomes. The combined use of VEGF/EGF/LL37 will likely result in enhanced wound healing. Pre-injection of TNT-capable nanowhiskers into the wound bed is expected to enable deeper gene delivery and lead to superior healing. While not the main subject of this project, electroceutical stimulation (e.g., pulsed electric fields) have been reported to improve wound outcomes. As such, studies with dressed wounds that are TNT′d with mock plasmids will allow us to determine if the electric field alone has a positive effect on healing.

qRT-PCR Measurements of Gene Expression

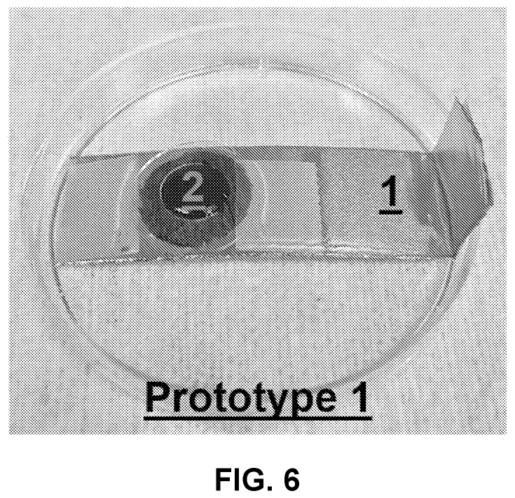

Nanofiber-based platforms were pre-loaded with pmaxGFP plasmids (core composed of polycaprolactone; shell composed of polycaprolactone/polyaniline). Transfection was conducted in the dermis of mice. An exemplary configuration is shown in FIG. 6, where 1 represents a negative electrode and 2 represents a reservoir containing the fiber-based platform, pre-soaked and pre-loaded with the plasmid solution. A positive counter-electrode was inserted into the skin to complete the circuit.

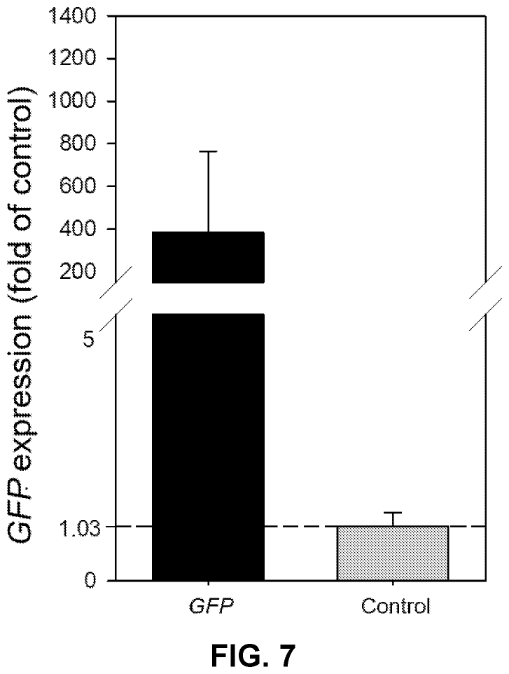

Skin was collected after at least 24 h post-transfection and analyzed by qRT-PCR. Quantitative analysis of gene expression indicates strong GFP overexpression (˜400-fold increase) compared to control skin (FIG. 7).

Immunofluorescence of the Skin after Transfection with pmaxGFP

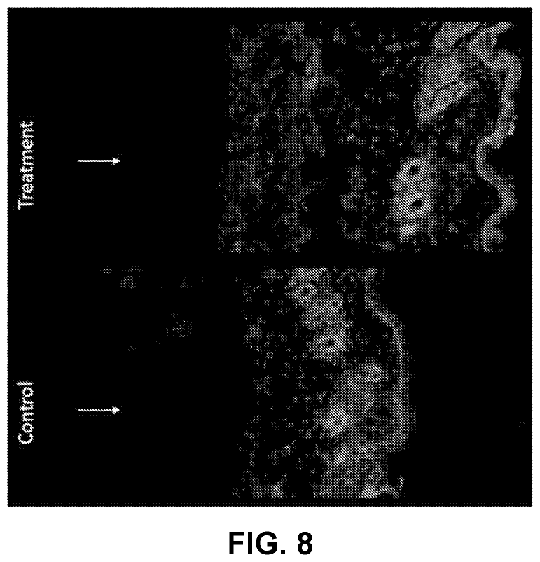

Immunofluorescence analysis of the transfected skin revealed stronger GFP immunoreactivity in the treated skin compared to the control (e.g., right-hand strip on treatment image, equivalent to epidermis, FIG. 8). A positive GFP signal was further detected deep in the skin in the hypodermal layer (left side of the images) of the treated skin. No clear GFP signal/immunoreactivity was detected in the control. Cell nuclei were stained and also appear in the treatment and control images.

Transfection Experiment with Genes that Induce Reprogramming into Islet-Like Tissue in the Skin

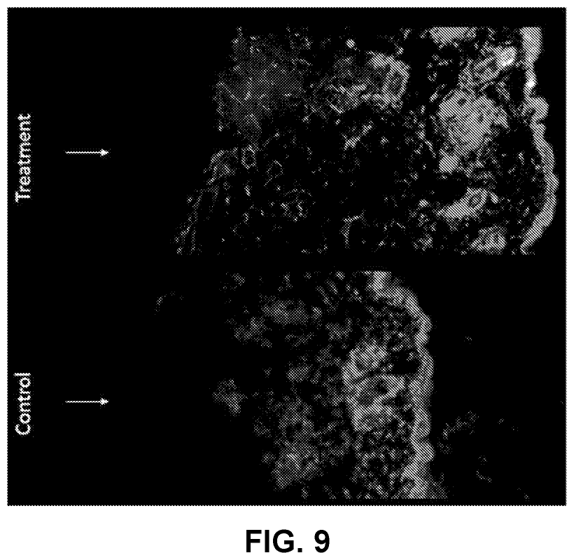

Immunofluorescence analysis of the transfected skin revealed stronger immunoreactivity for glucose sensor/transporter Glut2 (e.g., right-hand strip on treatment image, equivalent to epidermis, FIG. 9). A positive Glu2 signal was detected deep in the skin in the hypodermal layer (left side of the images). Baseline signal/immunoreactivity detected in the control is significantly weaker and less widespread compared to the treatment. Cell nuclei were stained and also appear in the treatment and control images.

Transfection Experiment with Genes that Induce Reprogramming into Neuron-Like Tissue in the Skin

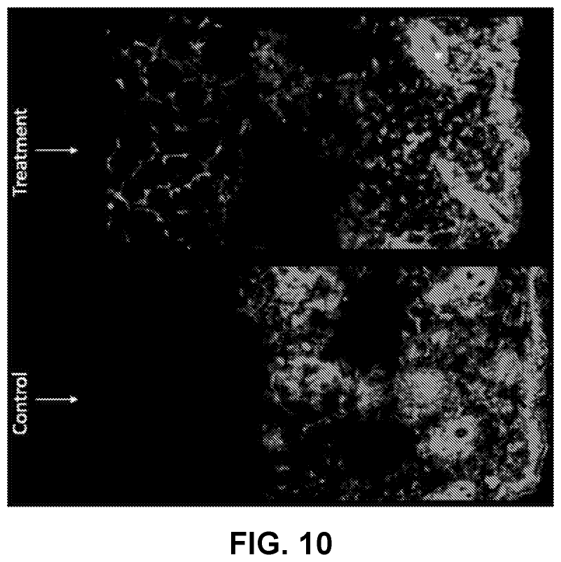

Immunofluorescence analysis of the skin revealed stronger immunoreactivity for neuronal marker Tuj1 in the epidermis (right side) and within hair follicles (*) (FIG. 10). Baseline signal/immunoreactivity detected in the control is markedly weaker compared to the treatment. Cell nuclei were stained and also appear in the treatment and control images.

Nanotransfection with Injected/Implanted Fiber Powders (Treated)

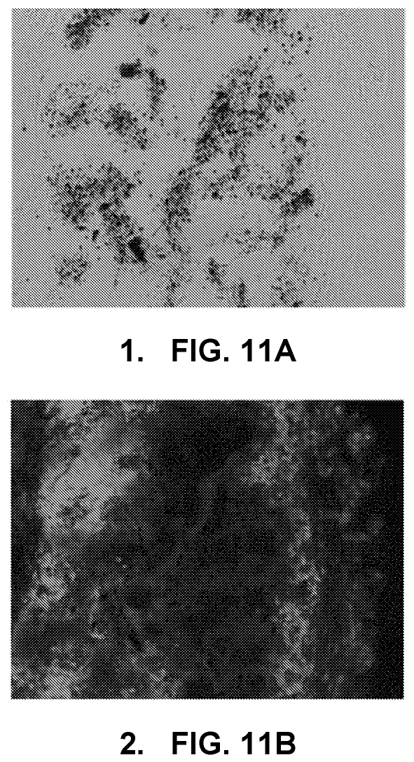

Powdered nanofibers as described elsewhere herein were pre-loaded with a plasmid of interest having a DDK flag/reporter. Pre-loaded fibers were injected into the flanks of mice with transfection being conducted 3 days post-implantation. Skin was biopsied and immunolabeled for the DDK flag. Fiber powder and corresponding DDK expression can be seen in FIGS. 11A and 11B, respectively.

Nanotransfection with Injected/Implanted Fiber Powders (Treated)

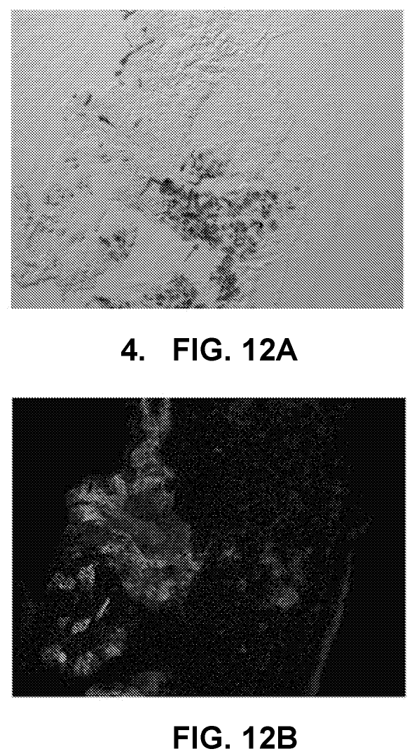

Control specimens were also injected with fibers that were not pre-loaded with any plasmid. Transfection and electroporation were conducted the same way as with treated/pre-loaded samples. Tissue was biopsied and immunolabeled for DDK flags; however, no discernable DDK expression can be seen in FIGS. 12A and 12B.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

What is claimed:

1. An electrospun core-shell fiber comprising:
(i) a central core that is electrically conductive having an exterior surface, wherein the core comprises a first polymer and an electroconductive material;
(ii) a shell adjacent to the exterior surface of the core, the shell comprising a second polymer; and
(iii) one or more bioactive agents in the shell.

2. The fiber of claim 1, wherein the electroconductive material comprises an electroconductive polymer, an electroconductive metal, or a combination thereof.

3. The fiber of claim 1, wherein the electroconductive polymer comprises polyaniline, polyaniline, a poly(pyrrole), an oxidized polyacetylene, a poly(fluorene), a polyphenylenes, a polypyrene, a polyazulene, a polynaphthalene, a poly(p-phenylene vinylene), a polycarbazole, a polyindoles, a polyazepine, a poly(thiophene), a poly(3,4-ethylenedioxythiophene), a poly(p-phenylene sulfide), a poly(naphthalene vinylene), a poly(3,4-ethylenedioxythiophene)-poly(styrenesulfonate), a poly(3,4-ethylenedioxythiophene)-block-poly(ethylene glycol), or any combination thereof

4. The fiber of claim 1, wherein the weight ratio of the first polymer to the electroconductive polymer is from 2:1 to 1:2.

5. The fiber of claim 1, wherein the electroconductive metal comprises tantalum, gold, niobium, silver, copper, aluminum, iron, zinc, molybdenum, lithium, nickel, palladium, platinum, tungsten, tin, rhodium, Iridium, or any combination thereof.

6. The fiber of claim 1, wherein the electroconductive metal comprises a plurality of metal nanoparticles.

7. The fiber of claim 1, wherein the weight ratio of the first polymer to the electroconductive metal is from 1:10 to 1:1.

8. The fiber of claim 1, wherein the electroconductive material comprises a combination of one or more electroconductive polymers and one or more electroconductive metals.

9. The fiber of claim 1, wherein the first polymer is biocompatible.

10. The fiber of claim 1, wherein the first polymer comprises a synthetic polymer comprising polyethylene terephthalate, a polyester, a polymethylmethacrylate, polyacrylonitrile, a silicone, a polyurethane, a polycarbonate, a polyether ketone ketone, a polyether ether ketone, a polyether imide, a polyamide, a polystyrene, a polyether sulfone, a polysulfone, a polycaprolactone (PCL), a polylactic acid (PLA), a polyglycolic acid (PGA), a polylactide-co-glycolide copolymer (PLGA), a polyglycerol sebacic, a polydiol citrate, a polyhydroxy butyrate, a polyether amide, a polydiaxanone, or any combination thereof.

11. The fiber of claim 1, wherein the first polymer comprises a natural polymer comprising fibronectin, collagen, gelatin, hyaluronic acid, chitosan, or any combination thereof.

12. The fiber of claim 1, wherein the first polymer comprises poly(e-caprolactone), polylactic acid (PLA), polyglycolic acid (PGA), polylactide-co-glycolide copolymer (PLGA), or any combination thereof.

13. The fiber of claim 1, wherein the core has an average diameter of about 100 nm to about 20 μm.

14. The fiber of claim 1, wherein the second polymer is biocompatible.

15. The fiber of claim 1, wherein the second polymer comprises a synthetic polymer comprising polyethylene terephthalate, a polyester, a polymethylmethacrylate, polyacrylonitrile, a silicone, a polyurethane, a polycarbonate, a polyether ketone ketone, a polyether ether ketone, a polyether imide, a polyamide, a polystyrene, a polyether sulfone, a polysulfone, a polycaprolactone (PCL), a polylactic acid (PLA), a polyglycolic acid (PGA), a polylactide-co-glycolide copolymer (PLGA), a polyglycerol sebacic, a polydiol citrate, a polyhydroxy butyrate, a polyether amide, a polydiaxanone, or any combination thereof.

16. The fiber of claim 1, wherein the second polymer comprises a natural polymer comprising fibronectin, collagen, gelatin, hyaluronic acid, chitosan, or any combination thereof.

17. The fiber of claim 1, wherein the second polymer comprises poly(e-caprolactone), polylactic acid (PLA), polyglycolic acid (PGA), polylactide-co-glycolide copolymer (PLGA), or any combination thereof.

18. The fiber of claim 1, wherein the first polymer and the second polymer are the same polymer.

19. The fiber of claim 1, wherein the first polymer and the second polymer are the different polymers.

20. The fiber of claim 1, wherein the shell has a thickness of about 10 nm to about 20 μm.

21. The fiber of claim 1, wherein the shell comprises a plurality of nanochannels.

22. The fiber of claim 1, wherein the nanochannels have an average diameter of about 10 nm to about 1,000 nm.

23. The fiber of claim 1, wherein the one or more bioactive agents comprise one or more of a nucleic acid, a peptide, a polypeptide, a small molecule, a vaccine, vesicles isolated from cells that have been reprogrammed, and any combination thereof.

24. The fiber of claim 1, wherein the one or more bioactive agents comprise one or more of genes such as LL37, laminin/collagen VII, and VEGF/EGF.

25. The fiber of claim 1, wherein the fibers are continuous.

26. A bioactive core-shell fiber produced by the method comprising contacting a core-shell fiber with a bioactive agent, wherein the core-shell fiber comprises:
(i) a central core that is electrically conductive having an exterior surface, wherein the core comprises a first polymer and an electroconductive material; and
(ii) a shell adjacent to the exterior surface of the core, the shell comprising a second polymer.

27. The fiber of claim 26, wherein the core-shell fiber is produced by electrospinning a concentric composition comprising an inner first composition comprising the first polymer and an electroconductive polymer and a second outer composition comprising the second polymer.

28. A fiber cluster comprising a plurality of claim 1.

29. The fiber cluster of claim 28, wherein the plurality of fibers are aligned or substantially aligned.

30. The fiber cluster of claim 28, wherein the plurality of fibers are randomly aligned.

31. The fiber cluster of claim 28, wherein fiber cluster has a fragment size of about 50 μm to about 500 μm.

32. The fiber cluster of claim 28, wherein the plurality of fibers comprise a plurality of long fibers and a plurality of short fibers.

33. A pharmaceutical formulation comprising a therapeutically effective amount of the fibers of any one of claim 1.

34. The pharmaceutical formulation of claim 33, wherein the formulation comprises a topical formulation.

35. The pharmaceutical formulation of claim 33, wherein the formulation is in the form of a cream, a balm, an ointment, a spray, a lotion, an emulsion, a gel, a salve, or a transdermal patch.

36. The pharmaceutical formulation of claim 33, wherein the formulation comprises a parenteral formulation.

37. The pharmaceutical formulation of claim 36, wherein the formulation is suitable for one or both of subcutaneous injection and intradermal injection.

38. A mat or mesh comprising a therapeutically effective amount of the fibers of claim 1.

39. The mat or mesh of claim 38, wherein the mat or mesh comprises the bioactive agent having different concentrations at different locations on the mat or mesh.

40. A wound dressing, gauge, or bandage comprising a therapeutically effective amount of the fibers of claim 1.

41. A textile or implant comprising a therapeutically effective amount of the fibers of claim 1.

42. A method for delivering a bioactive agent to a subject, the method comprising
(iii) administering or applying to the subject the fibers of any one of claim 1; and
(iv) applying an electric field to the fibers or fiber cluster.

43. The method of claim 42, wherein the method comprises administering to the subject the pharmaceutical formulation of any one of claims 33-37.

44. The method of claim 42, wherein the method comprises applying to the subject the mesh or mat of claim 38.

45. The method of claim 42, wherein the electric field has a voltage of from about 100 volts to about 500 volts.

46. The method of claim 42, wherein the electric field is applied continuously or is applied as a pulse.

47. The method of claim 42, wherein the subject has a wound, wherein the fibers or fiber cluster are applied at or near the wound.

48. The method of claim 47, wherein the wound comprises an acute wound or a chronic wound.

49. The method of claim 47, wherein the wound comprises a skin wound.

50. The method of claim 49, wherein the skin wound comprises a partial thickness wound or full thickness wound.

51. The method of claim 50, wherein the wound comprises a cut, laceration, surgical incision, puncture, graze, scratch, compression wound, abrasion, friction wound, decubitus ulcer; thermal effect wound, chemical wounds, wound caused by a pathogenic infections, ulcer, a diabetic-associated wound, skin graft/transplant donor and recipient sites, immune response condition, stomach or intestinal ulcer, oral wound, damaged cartilage or bone, amputation wound, or corneal lesion.

52. The method of claim 47, wherein the wound comprises nerve tissue damage.

53. The method of claim 47, wherein the wound comprises damage to a muscle, ligament, blood vessel, or eye.

54. The method of any one of claim 42, wherein the subject is in need of gene delivery.
